(19) United States
(12) Patent Application Publication
(10) Pub. No.: US 2026/0207651 A1
(43) Pub. Date: Jul. 23, 2026

(54) POLYDEOXYRIBONUCLEOTIDE (PDRN) DERIVED FROM PLANT CELLS

(71) Applicants: Jong Hwan Kim, Ridgefield, NJ (US); Sarfaraz K. Niazi, Deerfield, IL (US)

(72) Inventors: Jong Hwan Kim, Ridgefield, NJ (US); Sarfaraz K. Niazi, Deerfield, IL (US)

(21) Appl. No.: 19/029,829

(22) Filed: Jan. 17, 2025

(51) Int. Cl. A61K 31/711; A61K 8/60; A61K 8/9789; A61K 36/23; A61P 17/02; A61Q 17/04; A61Q 19/08

(52) U.S. Cl. CPC A61K 31/711; A61K 8/606; A61K 8/9789; A61K 36/23; A61P 17/02; A61Q 17/04; A61Q 19/08; A61K 2236/15; A61K 2236/19; A61K 2236/39; A61K 2236/53; A61K 2800/805; A61K 2800/82; A61K 2800/85

(57) ABSTRACT

The present invention discloses a manufacturing method for polydeoxyribonucleotide (PDRN), a DNA fragment, from plant sources, particularly Centella asiatica plant cells for use as an active ingredient in cosmetic, pharmaceutical, quasi-drug compositions, for skin improvement including protecting skin from ultraviolet radiation, strengthen skin barrier, moisturizing skin, reducing wrinkles, suppressing skin stress, regenerating skin, healing wounds, and slowing down skin aging.

### BACKGROUND OF THE INVENTION

Polydeoxyribonucleotide (PDRN) is generally used as a generic term to describe a mixture of DNA fragments within a specific molecular weight range, typically 50 to 1,500 kDa. In the medical and pharmaceutical context, PDRN refers to biologically active DNA fragments used in therapeutic applications, particularly for tissue repair, regeneration, and anti-inflammatory treatments. These fragments are characterized by their ability to stimulate cellular processes such as angiogenesis and collagen production. From a scientific and technical perspective, PDRN may simply denote a mixture of deoxyribonucleotide polymers derived from natural DNA sources, irrespective of their therapeutic utility, focusing on their molecular weight range and biochemical properties.

Polydeoxyribonucleotide (PDRN) is a compound composed of DNA fragments, typically extracted from the sperm of salmon species such as Oncorhynchus mykiss (salmon trout) or Oncorhynchus keta (chum salmon). These fragments, with molecular weights ranging from 50 to 1,500 kilodaltons, are widely recognized for their therapeutic applications. PDRN promotes tissue repair by activating adenosine A₂A receptors, which enhances cell proliferation and angiogenesis and exhibits anti-inflammatory properties that effectively treat conditions like diabetic foot ulcers and other chronic wounds. In skin care, PDRN improves skin elasticity, hydration, and texture and stimulates collagen production.

The mechanism of action of PDRN is twofold. First, it binds to adenosine A₂A receptors to reduce inflammation and promote tissue regeneration. Second, it provides nucleotides and nucleosides through the salvage pathway, aiding DNA synthesis and cellular proliferation. Clinical studies have shown PDRN to be highly effective in wound healing and aesthetic treatments, demonstrating excellent biocompatibility and minimal adverse reactions, making it a safe therapeutic option.

PDRN is a heterogeneous mixture of DNA fragments without a defined nucleotide sequence. Instead, its composition is well-characterized regarding fragment length and molecular weight. This diversity minimizes the risk of immune reactions due to the thorough removal of active proteins and peptides during purification. While salmon-derived PDRN is well-studied, PDRN derived from plants, which involves DNA fragments extracted from plant tissues such as leaves and seeds, differs in molecular composition and extraction process. Plant-derived DNA reflects the specific plant species' genome structure, often rich in methylated cytosines and unique repetitive elements. Although plant-derived PDRN produces DNA fragments of similar size, the structural and sequence differences in chromatin packaging may influence its therapeutic activity.

Aging skin presents one of the most visible and inevitable consequences of the passage of time, with intrinsic and extrinsic factors playing a role. Internally, reactive oxygen species (ROS) are produced as byproducts of metabolism, progressively damaging cellular structures and impairing skin function. Externally, prolonged exposure to ultraviolet radiation exacerbates this damage by triggering oxidative stress and inflammatory responses. These combined factors disrupt the skin's structural proteins, including collagen and elastin, leading to wrinkles, sagging, and reduced elasticity. PDRN effectively counters these processes by neutralizing the harmful effects of ROS. Activating specific signaling pathways reduces the expression of pro-inflammatory cytokines, inhibits the activity of MMPs responsible for collagen breakdown, and stimulates new collagen synthesis. These combined actions slow the signs of aging and restore the skin's functional integrity.

One of the most remarkable benefits of PDRN lies in its ability to stimulate tissue repair and accelerate wound healing. By activating adenosine receptors, PDRN significantly increases the production of VEGF, a growth factor essential for forming new blood vessels. This angiogenic effect ensures improved oxygenation and nutrient delivery to damaged tissues, which is crucial for recovery. Clinical and experimental studies have consistently demonstrated the effectiveness of PDRN in enhancing healing outcomes in a range of conditions, from chronic wounds to ischemic injuries. For instance, in burn injuries and other traumatic skin conditions, PDRN facilitates faster re-epithelialization and regeneration, contributing to more complete and aesthetically favorable recovery.

Collagen synthesis is another critical area where PDRN exerts a profound influence. Collagen is the skin's most abundant protein, serving as its structural backbone and providing strength and elasticity. Aging and environmental stressors lead to a decline in collagen production and an increase in its degradation. By stimulating adenosine receptors, PDRN downregulates inhibitory pathways and upregulates factors that promote collagen synthesis. This process enhances skin elasticity, reduces the appearance of fine lines and wrinkles, and restores the skin's overall firmness. PDRN's ability to rebuild the extracellular matrix also helps prevent the loss of structural integrity that typically accompanies aging.

In addition to its role in tissue regeneration, PDRN exhibits potent anti-pigmentation effects. Hyperpigmentation, often caused by prolonged exposure to sunlight or chronic inflammation, results in disorders such as melasma and post-inflammatory hyperpigmentation. PDRN directly inhibits the enzyme tyrosinase, which is central to melanin production. By suppressing the genetic and enzymatic pathways involved in melanogenesis, PDRN effectively reduces melanin synthesis and accumulation. This makes it an attractive agent for skin-whitening applications and the management of pigmentation disorders. However, further studies must fully elucidate its mechanisms and optimize its use.

Beyond its dermatological applications, PDRN has shown promise in addressing hair loss. Pattern hair loss, characterized by progressive thinning and follicular miniaturization, affects a large population segment and can have a significant psychosocial impact. Studies suggest that PDRN injections, especially when combined with platelet-rich plasma (PRP) or laser therapy, improve hair health by increasing follicular activity. These treatments have been shown to enhance hair thickness and, to a lesser extent, hair density, offering a valuable option for individuals seeking non-surgical solutions for hair regeneration.

The administration of PDRN is tailored to ensure maximum efficacy by delivering it directly to the dermal layer of the skin, where fibroblasts, the primary drivers of collagen production, are located. Techniques such as microneedling, fractional laser application, and mesotherapy enable deeper penetration of PDRN into the target tissues. By bypassing the skin's natural barrier, these methods ensure that PDRN reaches the dermal layer, where it can exert its regenerative and anti-aging effects most effectively. Unlike traditional therapies that rely on hyaluronic acid (HA) for hydration and volume, PDRN focuses on stimulating tissue repair and revitalization. Its ability to work at the cellular level distinguishes it as a revolutionary approach to skin rejuvenation.

Safety is a cornerstone of PDRN's therapeutic profile. Extensive studies have confirmed its biocompatibility and lack of immunogenicity, critical for its use in medical and cosmetic applications. PDRN is extracted and purified through processes that eliminate impurities, ensuring a highly pure final product. Preclinical and clinical studies have shown that PDRN is well-tolerated, with no observed toxicity in vital organs or adverse effects on long-term use. This safety profile further reinforces its suitability for widespread application.

While the term is broadly applied, distinctions based on molecular weight can arise depending on the context or intended application. In some instances, molecular weight-specific classifications are used to differentiate DNA fragments. For example, high-molecular-weight fragments (close to 1,500 kDa) may be highlighted for their structural properties. In comparison, low-molecular-weight fragments (around 50-300 kDa) may be preferred for applications requiring enhanced cellular uptake or bioavailability. While important in research or technical specifications, these distinctions do not typically result in unique names, and the term PDRN remains the generic descriptor. In regenerative medicine, all DNA fragments within the typical molecular weight range are grouped under PDRN and defined primarily by their bioactive properties rather than specific sizes. Similarly, in cosmetics, while some products may emphasize fragment size in marketing to suggest targeted effects or better absorption, they are still marketed under the generic term PDRN.

Researchers often differentiate between DNA fragment sizes in scientific research when studying specific effects or mechanisms, though they do not assign unique names to these fragments. Regulatory bodies, on the other hand, classify PDRN based on production processes, purity, and biological activity rather than strictly on molecular weight, keeping the term generic unless a specific product or derivative is trademarked or branded with a proprietary name. Therefore, while molecular weight distinctions may be necessary in technical or research contexts, PDRN remains a broad term encompassing DNA fragments within a defined size range.

The PDRN used in skin treatments generally has a specific length, corresponding to DNA fragments within a defined molecular weight range. PDRN typically consists of DNA fragments with molecular weights ranging between 50 and 1500 kDa for cosmetic and therapeutic applications, including skin repair and rejuvenation. This range translates to DNA fragments of approximately 100-3000 base pairs in length. The specific length is chosen because it is bioactive and optimally sized for promoting cellular processes such as tissue regeneration, angiogenesis, and collagen synthesis.

These DNA fragments are small enough to be absorbed or taken up effectively by cells and exert their biological effects, yet large enough to retain their structural integrity and activity. In skin treatments, the PDRN interacts with cell receptors, promotes wound healing, and enhances skin regeneration by activating pathways involved in tissue repair, such as stimulating fibroblast activity and improving extracellular matrix production.

Manufacturers typically optimize the PDRN molecular weight range for skin applications to ensure effective penetration, safety, and therapeutic outcomes. Products used in aesthetic medicine, such as mesotherapy solutions or injectables for skin rejuvenation, often include PDRN fragments within this size range. This ensures the DNA fragments have the bioavailability and activity necessary to support skin healing and anti-aging effects

The definition of PDRN can also vary based on its source. For instance, when derived from animal tissues like salmon sperm, PDRN is typically described as purified and processed nucleotides designed to retain bioactivity. At the same time, the definition of plant sources may be broader and depend on the specific DNA extraction and fragmentation processes.

Although PDRN has proven its efficacy as an animal-derived ingredient extracted from salmon sperm, it is unsuitable for ‘vegan’ products that do not contain animal ingredients and ‘cruelty-free’ cosmetics that have not undergone animal testing. Vegan certification is only granted to products that do not use any ingredients obtained from animals, which has the advantages of being less irritating to the skin and protecting animals. Therefore, methods to extract PDRN from plant sources instead of animals have been attempted, but there have been few visible results yet.

Plants do not contain PDRN, but they all contain DNA that can be used to construct PDRN, but the choice of plants that can yield commercially viable PDRN supply. Young, rapidly growing tissues such as shoots, meristems, and sprouts from plants like wheatgrass (Triticum aestivum), barley (Hordeum vulgare), and maize (Zea mays) are ideal due to their high nucleic acid concentration. Seeds, particularly during germination, are also excellent sources; examples include soybeans (Glycine max), lentils (Lens culinaris), and rice (Oryza sativa). Microalgae and algae like Spirulina and Chlorella vulgaris stand out for their high nucleic acid content and ease of DNA extraction due to the absence of rigid cell walls. Medicinal plants such as Panax ginseng (ginseng) and Aloe vera are promising for their therapeutic potential and DNA content. Leafy vegetables with high biomass, such as spinach (Spinacia oleracea) and lettuce (Lactuca sativa), are economical options for DNA extraction.

Additionally, specialized crops with minimal secondary metabolites, like tobacco (Nicotiana tabacum) and potato (Solanum tuberosum), provide high DNA yields and are commonly used in molecular biology. Microalgae like Spirulina and Chlorella are considered the best overall choice due to their high nucleic acid density and ease of processing. At the same time, seeds and medicinal plants like Panax ginseng are valuable for their dual role in DNA provision and biological activity. Broccoli (Brassica oleracea) is another practical plant source of significance. Additionally, roses (Rosa spp.) are being explored for their potential in PDRN extraction, although specific studies are still limited.

Additionally, advances in synthetic biology offer the potential to synthesize PDRN without relying on biological tissue, which could circumvent the need for plant or animal sources entirely. For an effective exploration of plant-based PDRN production, high-yield DNA. Regardless of the plant source, extracting DNA and converting it into PDRN remains a novelty, as disclosed in the present invention.

### SUMMARY OF INVENTION

The term “PDRN” of the invention refers to polydeoxyribonucleotide, a mixture of the short deoxyribonucleotide. That is, PDRN is a low molecular weight DNA complex made by fragmenting a DNA chain into a certain size, and the one used in the present invention may have a size of 300 bp or less and a molecular weight of 74 kDa or less. The term “PDRN” may be a type of nucleic acid fragment.

The term “plant cell” of the present invention refers to a cell mass produced by culturing tissue cut from a plant in a medium, and in a broad sense, includes callus, which is a cell mass that has lost the stress that causes normal organ formation or tissue differentiation, or plant tumor tissue produced by infection with Agrobacterium, etc.

The term “callus” of the present invention is an unorganized mass of plant laticiferous cells, a tissue created when a plant is wounded. In the present invention, it can be the callus in which the callus is induced from stem, root, leaf, berries, flower, or a combination of the plant, but it is not limited to it. The method for inducing the callus of the invention is not limited; it can be extracted according to the general method in the technical field.

One exemplary object of the present invention is to provide a method for producing polydeoxyribonucleotide (PDRN) from plant cells, including the following proprietary steps.

Choosing a plant from Aloe vera, broccoli (Brassica oleracea), Panax ginseng (Korean ginseng), roses, young rapidly growing tissues like shoots, meristems, and sprouts, microalgae such as Spirulina and Chlorella, legumes such as soybeans and lentils, or any other such plant source. Typical manufacturing steps are shown in FIG. 1.

The present invention provides cosmetic compositions, including PDRN as an active ingredient for skin improvement.

Another exemplary object of the present invention is to provide pharmaceutical compositions including PDRN as an active ingredient for the prevention or treatment of one skin disease selected from the group of diseases consisting of skin disease, psoriasis, skin wound, skin scar by the ultraviolet ray and the skin inflammation.

Another exemplary object of the present invention is to provide quasi-drug compositions, including PDRN as an active ingredient for skin improvement.

Another exemplary object of the present invention is to provide compositions, including PDRN as an active ingredient for skin improvement.

Another exemplary object of the present invention is to provide feed compositions, including PDRN as an active ingredient for skin improvement.

The technical problems in manufacturing PDRN in the invention disclosed in this specification are not limited to the resolution of the problems mentioned above, and other issues not mentioned will be clearly understood by those skilled in the art from the following description.

### DETAILS OF THE INVENTION

Unlike the PDRN derived from salmon, extracting PDRN from plant sources presents unique challenges that must be addressed. The rigid cell walls of plant cells, composed of cellulose, hemicellulose, and lignin, pose significant obstacles to efficient DNA extraction. Additionally, plants produce secondary metabolites such as polyphenols and polysaccharides, which can interfere with extraction and purification. Achieving high yields of pure PDRN requires optimized protocols to overcome these barriers and ensure the final product meets therapeutic standards. Currently, no widely adopted commercial processes for producing PDRN from plants exist. However, ongoing research and advancements in biotechnology and molecular biology techniques hold promise for overcoming these challenges and establishing viable methods for plant-based PDRN production. Given the rapid pace of development in this field, consulting recent scientific literature and industry reports is essential for the most current insights. Against this background, the inventors have completed the present invention by developing a method to produce high-purity PDRN from plant cells and confirming that PDRN produced by this method has wrinkle improvement, wound healing, and anti-aging effects.

Extracting DNA from plant cells and converting it into Polydeoxyribonucleotide (PDRN) involves a multi-step process that begins with isolating DNA from plant tissues and processing it into smaller fragments of specific molecular weights.

In the present invention, the plant cell is obtained by culturing a part of a plant, such as a leaf, stem, root, or a part thereof, and includes a culture solution in which a part of the plant is cultured.

In another embodiment, the present invention provides cosmetic compositions for skin improvement, including PDRN as an active ingredient.

In the present invention, “skin improvement” means any act of improving or benefiting skin by using PDRN produced by the production method of the present invention.

In the present invention, skin improvement may include skin protection from ultraviolet rays, strengthening of the skin barrier, moisturizing, wrinkle improvement, suppression of skin stress, skin regeneration, wound healing, or anti-aging.

The adequate amount of the application means the invention mentioned above was applied to achieve skin improvement.

The cosmetic composition of the present invention can be manufactured in various forms. For example, cosmetic compositions may be manufactured in any formulation commonly manufactured in the industry. They can be a solution, suspension, oil emulsion, paste, gel, cream, lotion, powder, soap, cleansing, oil, powder foundation, emulsion foundation, wax foundation spray, etc., but it is not limited to it. More specifically, it can have the formulation selected from the group consisting of skin lotion, skin softener, skin toner, astringent, lotion, milk lotion, moisture lotion, nutritive lotion, massage cream, moisturizing cream, moisture cream, hand cream, nutritious essence, pack, soap, shampoo, cleansing foam, cleansing lotion, cleansing cream, body lotion, body cleanser, emulsion, lipstick, makeup base, foundation, press powder and loose powder but it is not limited to it.

The cosmetic composition of the present invention may contain acceptable carriers in cosmetic formulations in addition to its active ingredient. “Acceptable carriers in cosmetic formulations” refers to a compound or composition that is already known and used or a compound or composition to be developed in the future that can be included in cosmetic formulations and that does not have toxicity, instability, or irritation that is more than what the human body can adapt to when in contact with the skin. The carrier may be included in the cosmetic composition of the present invention in an amount of about 1 wt % to about 99.99 wt % of the total weight of the composition, preferably about 5 wt % to about 99 wt % of the composition weight.

However, the above ratio may vary depending on the form of the cosmetic being manufactured and its specific application scope (such as face or hands) or desired application amount. Thus, the ratio should not be understood as limiting the scope of the present invention in any way.

Meanwhile, the carrier may include alcohol, oil, surfactant, fatty acid, silicone oil, humectant, moisturizer, viscosity modifier, emulsion, stabilizer, sunscreen, colorant, fragrance, etc. Compounds or compositions that can be used as alcohol, oil, surfactant, fatty acid, silicone oil, humectant, moisturizer, viscosity modifier, emulsion, stabilizer, sunscreen, colorant, and fragrance are already known in the art, and those skilled in the art can select and use appropriate substances or compositions.

Another aspect of the present invention provides pharmaceutical compositions for preventing or treating skin diseases selected from the group consisting of skin diseases caused by ultraviolet rays, psoriasis, skin wounds, skin scars, and skin inflammation, including PDRN as an active ingredient.

In the present invention, the skin diseases caused by ultraviolet rays may be one or more selected from the group consisting of sunburn, erythema, edema, pigmentation, skin inflammation, skin keratosis, photoallergy, phototoxicity, photosensitization, and photoaging.

In the present invention, “prevention” means any act of improving skin diseases by administering the pharmaceutical composition of the present invention, and “treatment” means any act of improving or alleviating the developed skin disease or changing it beneficially by administering the pharmaceutical composition of the present invention. In the present invention, “improvement” means improving skin diseases by administering the composition.

In the present invention, “pharmaceutical composition” means a composition manufactured to prevent or treat diseases, and it can be formulated in various forms according to conventional methods. For example, it can be formulated into oral formulations such as powders, granules, tablets, capsules, suspensions, emulsions, syrups, etc., and formulated into topical agents and sterile injectable solutions. Specifically, the administration route may be appropriate, including topical, oral, intravenous, intramuscular, and direct absorption through mucosal tissues. Two or more routes may be combined. An example of a combination of two or more routes is when two or more formulations of drugs are combined according to the administration route; for example, one drug is administered intravenously first, and another is administered topically second.

The pharmaceutical composition of the present invention may further include pharmaceutically acceptable carriers, excipients, or diluents according to conventional methods. Pharmaceutically acceptable carriers are well known in the art depending on the route of administration or formulation, and specific examples can be found in the pharmacopeias of each country, including the ‘Korean Pharmacopoeia.’ Carriers, excipients, and diluents that may be included in the compositions of the present invention include but are not limited to, lactose, dextrose, sucrose, sorbitol, mannitol, xylitol, erythritol, maltitol, starch, acacia gum, alginate, gelatin, calcium phosphate, calcium silicate, cellulose, methylcellulose, microcrystalline cellulose, polyvinyl pyrrolidone, water, methyl hydroxybenzoate, propyl hydroxybenzoate, talc, magnesium stearate, and mineral oil. In addition, carriers, excipients, and diluents that may be included in the compositions of the present invention may be non-natural carriers but are not limited to it.

The pharmaceutical composition of the present invention can be formulated into oral formulations such as powders, granules, tablets, capsules, suspensions, emulsions, syrups, aerosols, topical agents, suppositories, sterile injectable solutions, as well as in a nanoparticle formulation.

Specifically, when formulating, it can be prepared using diluents or excipients such as fillers, extenders, binders, humectants, disintegrants, surfactants, etc., which are commonly used. Solid formulations for oral administration include tablets, pills, powders, granules, capsules, etc., and these solid formulations can be prepared by mixing the compound with at least one excipient, for example, starch, calcium carbonate, sucrose, lactose, gelatin, etc. In addition to simple excipients, lubricants such as magnesium stearate and talc can also be used.

Liquid formulations for oral administration include suspensions, internal solutions, emulsions, syrups, etc. In addition to simple diluents such as water and liquid paraffin, various excipients such as humectants, sweeteners, flavoring agents, and preservatives can be included. Formulations for non-oral administration include sterile aqueous solutions, non-aqueous solvents, suspensions, emulsions, freeze-dried forms, and suppositories.

Non-aqueous solvents and suspensions include vegetable oils such as propylene glycol, polyethylene glycol, olive oil, and injectable esters such as ethyl oleate. Bases for suppositories include Witepsol, macrogol, tween 61, cacao butter, laurin butter, and glycerogelatin. The specific formulation of the pharmaceutical composition is well known in the art, and reference can be made to literature such as [Remington's Pharmaceutical Sciences (19th ed. 1995)]. The literature is considered part of this specification.

The pharmaceutical composition of the present invention is administered in a pharmaceutically effective amount. The pharmaceutically effective amount means an amount sufficient to treat the disease at a reasonable benefit/risk ratio applicable to medical treatment without causing side effects, and effective dose levels may be determined based on factors including the patient's health status, type and severity of the disease, activity of the drug, sensitivity to the drug, method of administration, time of administration, route of administration and excretion rate, duration of treatment, combination or concurrent use of drugs, and other factors well known in the medical field. The dosage and frequency of administration do not limit the scope of the present invention in any way.

The pharmaceutical composition of the present invention can be administered to mammals such as mice, dogs, cats, cows, horses, pigs, and humans through various routes, and it may be desirable for humans. All administration methods can be anticipated; for example, it can be administered orally, intravenously, intramuscularly, or subcutaneously, but it is not limited to it.

In the present invention, “including as an active ingredient” means including an adequate amount to exhibit preventive or therapeutic effects on skin diseases as a pharmaceutical composition.

Another aspect of the present invention is that it provides a quasi-drug composition for skin improvement, including PDRN as an active ingredient.

The terms “PDRN” and “skin improvement” in the present invention are as described above.

The quasi-drug composition of the present invention may include ingredients commonly used in quasi-drug compositions in addition to PDRN, for example, abrasives, humectants, binders, foaming agents, sweeteners, preservatives, active ingredients, flavoring agents, colorants, solvents, whitening agents, solubilizers, or pH adjusters, but are not limited to it.

The pharmaceutical composition of the present invention may be exemplified as an external preparation, powder, sterilizing disinfectant, toothpaste, ointment, lotion, internal preparation (vitamin/mineral preparation, nutritious tonic), wet tissue, spray patch, band or patch, etc., but is not particularly limited to it. The pharmaceutical composition's formulation method, dosage, method of use, composition, etc., may be appropriately selected by a person skilled in the art from standard techniques known in the relevant technical field.

The terms “PDRN” and “skin improvement” in the present invention are as described above.

The composition of the present invention can be manufactured in various formulations. Unlike general medicines, it has the advantage of not having side effects that may occur with long-term use of medicines. The composition of the present invention can be manufactured in any form. Specifically, it can be one or more formulations selected from the group consisting of health-functional formulations such as tablets, capsules, pills, granules, liquids, powders, flakes, pastes, syrups, gels, jellies, bars, beverages, gums, and candies, but is not particularly limited to it.

In the present invention, the term “active ingredient” includes an ingredient that exhibits activity for its purpose or can exhibit activity together with an inactive carrier.

In addition to PDRN, an active ingredient, the composition of the present invention may contain a compound or natural extract known to have a skin improvement effect to increase or enhance the skin improvement effect.

In the present invention, the active ingredient may be included in any amount (effective amount) depending on the intended use, formulation, mixing purpose, etc., if it can exhibit skin improvement activity. A typical effective amount may be included within a range of 0.001 wt % to 99.99 wt % based on the total weight of the composition. The “effective amount” herein refers to the amount of the active ingredient that can induce a skin improvement effect. This effective amount can be experimentally determined within the normal ability range of a person skilled in the art.

Another aspect of achieving the above objective is that the present invention provides a skin improvement feed composition containing PDRN as an active ingredient.

The present invention's terms “PDRN” and “skin improvement” are described above.

The above feed composition may include a feed additive.

Another aspect of achieving the above objective is that the present invention provides a skin improvement method, including administering adequate PDRN to a needy subject.

In the present invention, “effective amount” means an amount sufficient for the composition of the present invention described above to achieve skin improvement efficacy.

The term “subject” as used herein includes but is not limited to, for example, humans, monkeys, cows, horses, sheep, pigs, chickens, turkeys, quails, cats, dogs, mice, rats, rabbits, or guinea pigs, and preferably means mammal, more preferably humans.

The route of administration of the composition of the present invention may be oral or parenteral administration via any standard route if it can reach the target tissue. In addition, the composition of the present invention may be administered using any device capable of delivering the active ingredient to the target cell, tissue, or organ.

Another aspect of the present invention for achieving the above object is using PDRN for skin improvement.

### Efficacy Testing of the Invention

The present invention can produce high-purity and high-concentration PDRN from plant cells. The plant-derived PDRN produced accordingly has the effects of protecting skin from ultraviolet irradiation, strengthening the skin barrier, moisturizing, improving wrinkles, suppressing skin stress, regenerating skin, and healing wounds or anti-aging, so it can be usefully used as a cosmetic composition for improving skin.

Cultivation of Centella asiatica Plant Cells

The leaves of Centella asiatica were immersed in 70% ethanol for 30 seconds, washed with sterile water, and then disinfected with a solution of 30% bleach and Tween 20 for 20 minutes. After washing three times with sterile water, the leaves were wounded with a sharp knife and cultured in a basic MS medium (Murashige and Skoog 1962, Duchefa, Cat No. M0221) containing 1 mg/L Zeatin, 3% Sucrose, and 8 g/L agar at 25° C. and 70% humidity in the dark to induce cell formation. The plant cells induced in solid medium were subcultured at two-week intervals, and liquid mass culture was performed in a 20 L bioreactor (purchased from Samsung Science) under culture room conditions of 23±2° C. and 70% humidity, with an air supply of approximately 0.1 vvm, for 5 weeks, and harvested to be used for PDRN isolation.

PDRN Obtained from Centella asiatica Plant Cells
Ethanol Immersion: Removal of Chlorophyll from Plant Cells Themselves

The harvested 2 kg FW of Centella asiatica plant cells was placed on a filter paper (miracloth filter 0.22 to 0.25 m) and immersed in 95% alcohol ethanol for 1 to 2 hours.

### Cell Disruption

The decolorized Centella asiatica cells were stored at −20 to −80° C. The first disruption occurred as the frozen cells thawed naturally, and the second disruption was performed by blending with Lysis Buffer (0.5M sodium chloride, Disodium Cocoyl Glutamate). For sufficient cell disruption, Centella asiatica cells were sufficiently reacted in Lysis Buffer (0.5 M sodium chloride, Disodium Cocoyl Glutamate) at 65° C. for 60 minutes, followed by Centella asiatica plant cell physical disruption for 30 minutes to 1 hour at a high radio frequency of 150 to 200 kHz.

### Protein Removal

Endo- and Exo-Protease were added to remove plant proteins and allowed to react at room temperature for 30-60 minutes. After sufficient reaction, the first impurities and cell debris were removed by filtration and the second by centrifugation, retaining only the supernatant.

### Securing DNA

Add 95% ethanol in the same volume as the supernatant and precipitate at −20 to −80° C. for 12 hours. Then, a centrifuge is used to obtain the settled DNA pellet and discard the supernatant. Add 80% ethanol to the remaining DNA pellet and perform two washing steps. Additional centrifugation is performed to remove supernatant impurities other than the DNA pellet. After completely drying the DNA pellet at 40 to 65° C. for 6 to 12 hours, purified water is added to obtain DNA from the DNA pellet.

### Securing PDRN

The extracted DNA was treated with an ultrasonic cleaner (28 kHz) for 90-120 minutes to obtain low molecular weight DNA (PDRN) of 100-200 bp (A260/A280=1.8-2.0). The secured PDRN is stored in a contamination-preventing state after passing through a 0.45 m to 0.22 m filter stage.

The present invention is completed with the PDRN material secured from the Centella asiatica plant cell secured in this way.

### Experimental Example 1: Test for Non-Irritating Effect on Skin Cells

To confirm the cytotoxicity and skin irritation of the composition according to the present invention on skin cells, a CCK-8 assay was conducted.

Specifically, HaCaT (Human Keratinocyte) cells were dispensed into a 96-well plate with DMEM (Dulbecco Modified Eagle Medium) containing 10% FBS (Fetal Bovine Serum) and 1% Antibiotic-Antimycotic, and cultured for 24 hours in an incubator at 37° C. with 5% CO₂. After culturing, the control group, the composition of the present invention, and salmon PDRN (Millipore, Cat #262012) were treated on the cells and further cultured for 24 hours. After removing the culture medium, D-Plus™ CCK cell viability assay solution was diluted 10 times with medium without FBS and added 100 uL per well, reacting for 3 hours. The absorbance was then measured at 450 nm (Table 1)

TABLE 1
HaCaT Cell Viability (%) derived from different sources

| | | HaCaT |
|---|---|---|
| | Treatment | Cell |
| | Concentration | Viability |
| Category | (ppm) | (%) |
| Control | — | 100 |
| Centella Asiatica Cell PDRN | 0.5 | 117.7 |
| | 1 | 132.2 |
| | 5 | 136.9 |
| | 10 | 126.0 |
| Salmon PDRN | 0.5 | 126.9 |
| | 1 | 122.5 |
| | 5 | 122.1 |
| | 10 | 138.1 |
| Lotus Cell PDRN | 1 | 107.1 |
| Green Tea Cell PDRN | | 122.1 |
| Soybean Cell PDRN | | 126.8 |
| Daffodil Cell PDRN | | 116.1 |
| Pine Tree Cell PDRN | | 105.1 |
| Rice Cell PDRN | | 108.9 |
| Echinacea Cell PDRN | | 113.2 |
| Ginseng Adventitious Root | | 119.5 |
| PDRN | | |

As shown in Table 1 and FIG. 1, both the composition of the present invention and salmon PDRN, when treated at concentrations of 0.5 to 10 ppm, did not show toxicity in keratinocytes but increased cell viability. Therefore, it was confirmed that the composition of the present invention is a safe substance with a low possibility of causing skin irritation.

### Experimental Example 2: Protective Effect on Skin Cells from UV Irradiation

The light source used for UV irradiation was an Ultraviolet crosslinker CL-1000 (Ultra-Violet Products, CA) emitting UV at a wavelength of 302 nm. HaCaT cells (human keratinocytes) were dispensed into a 96-well plate and cultured in an incubator for 24 hours. After removing the medium, it was replaced with a fresh cell culture medium without FBS, and starvation was performed for 4 hours. After washing with phosphate-buffered saline, the cells were exposed to UV at 20 mJ/cm², followed by additional culturing for 24 hours with the composition of the present invention and salmon PDRN. Then, [Experimental Example 1] was performed (Table 2).

TABLE 2
Comparative protective effect against UV radiation

| | | | Protective |
|---|---|---|---|
| | | | Effect |
| | | Treatment | Against UV |
| | UV | Concentration | Irradiation |
| Category | Irradiation | (ppm) | (Viability, %) |
| Control | − | − | 100 |
| Negative Control | + | − | 49.8 |
| Centella Asiatica Cell | | 0.5 | 72.0 |
| PDRN | | 1.0 | 69.2 |
| | | 5.0 | 58.9 |
| | | 10.0 | 60.3 |
| Salmon PDRN | | 0.5 | 58.4 |
| | | 1.0 | 58.6 |
| | | 5.0 | 58.5 |
| | | 10.0 | 58.7 |
| Lotus Cell PDRN | | 1 | 55.1 |
| Green Tea Cell PDRN | | | 58.2 |
| Soybean Cell PDRN | | | 63.1 |
| Daffodil Cell PDRN | | | 61.4 |
| Pine Tree Cell PDRN | | | 53.5 |
| Rice Cell PDRN | | | 64.1 |
| Echinacea Cell PDRN | | | 67.3 |
| Ginseng Adventitious | | | 68.1 |
| Root PDRN | | | |

As shown in Table 2 and FIG. 2, cell viability increased compared to the control group when treated with Centella asiatica cell-derived PDRN and salmon PDRN. Notably, the composition of the present invention showed significantly higher viability at low concentrations (0.5, 1 ppm) compared to other concentration ranges. Therefore, it was confirmed that the composition of the present invention has a protective effect on skin cells from UV irradiation.

### Experimental Example 3: Evaluation of Wound Healing Efficacy in Skin Cells

When the skin is wounded, skin cells divide to fill the tissue loss and regenerate the damaged skin tissue to its original state. However, as aging occurs, epidermal cells' division and regeneration rate decreases by about half, weakening the wound healing ability and reducing the skin's immune function. To confirm the wound healing efficacy of the composition of the present invention, a scratch was made on a cell monolayer, and the test sample was treated to measure cell mobility over time, evaluating skin regeneration efficacy.

Specifically, HaCaT cells were dispensed into a 24-well plate and cultured for 24 hours. Then, a scratch was made using an SPLScar™ Scratcher, and cell images at hour 0 were taken with a microscope. The control group, the composition of the present invention, and salmon PDRN were treated and further cultured for 20-24 hours. After 20-24 hours, cell images were taken with a microscope, and the degree of wound healing was measured by calculating the cell migration area using the equation: Healing area (%) (Surface area at 24 hours/Surface area at hour 0)×100 (Table 3).

TABLE 3
Comparative Healing Area

| | Treatment | Healing |
|---|---|---|
| | Concentration | Area |
| Treatment Sample | (ppm) | (%) |
| Control Group (0 hours) | — | 0 |
| Control Group | — | 45.3 |
| Positive Control Group | EGF 100 ng/mL | 76.1 |
| Centella Asiatica Cell PDRN | 1 | 59.0 |
| Salmon PDRN | | 55.1 |
| Lotus Cell PDRN | | 58.1 |
| Green Tea Cell PDRN | | 56.2 |
| Soybean Cell PDRN | | 57.3 |
| Narcissus Cell PDRN | | 54.2 |
| Pine Cell PDRN | | 56.6 |
| Rice Cell PDRN | | 54.1 |
| Echinacea Cell PDRN | | 56.7 |
| Ginseng Adventitious Root PDRN | | 62.5 |

The composition of the present invention showed a significantly similar skin wound healing rate to salmon PDRN compared to the control group. Therefore, it was confirmed that the composition of the present invention is effective in wound healing.

### Experimental Example 4: Evaluation of Anti-Aging Efficacy Using SA-p-gal Staining

Cells were dispensed into 12-well plates (Keratinocyte; 5×10⁵/well, Fibroblast, Melanocyte; 5×10⁴/well) and cultured under cell culture conditions for 24 hours. After that, the samples were treated with the cells and cultured for 24 hours, followed by SA-3-gal Staining.

Specifically, after removing the medium, each well was washed with phosphate buffer, then 0.5 ml of fixative was added to each well and reacted at room temperature for 2 hours. After removing the solution, 0.5 ml of Staining solution mix was added to each well, covered, and reacted at 37° C. for 1 to 24 hours. Then, images were obtained under a microscope, and the number of cells showing a positive reaction to the stain was counted and compared (Table 4).

TABLE 4
Comparative Number of β-gal Staining Positive Cells

| | | | Number |
|---|---|---|---|
| | | | of β-gal |
| | | Treatment | Staining |
| | | Concentration | Positive |
| Passage | Treatment Sample | (ppm) | Cells |
| 8 | Control Group | — | — |
| 61 | Control Group | — | 103.7 |
| | Centella Asiatica Cell | 1 | 63.8 |
| | PDRN | | |
| | Salmon PDRN | | 82.9 |
| | Lotus Cell PDRN | | 80.1 |
| | Green Tea Cell PDRN | | 68.5 |
| | Soybean Cell PDRN | | 75.1 |
| | Narcissus Cell PDRN | | 71.2 |
| | Pine Cell PDRN | | 69.7 |
| | Rice Cell PDRN | | 68.2 |
| | Echinacea Cell PDRN | | 63.0 |
| | Ginseng Adventitious Root | | 61.2 |
| | PDRN | | |

As shown in [Table 4, FIG. 4], it was confirmed that the number of cells showing a positive reaction to 3-gal decreased in the composition of the present invention and salmon PDRN compared to the control group. In particular, the composition of the present invention showed a lower number of p-gal staining positive cells than salmon PDRN, confirming that only the composition of the present invention has an anti-aging effect compared to salmon PDRN and the control group.

### Experimental Example 5: Evaluation of Wrinkle Improvement, Barrier Enhancement, and Antioxidant Efficacy in Skin Cells by Gene Expression

Cells were dispensed into a 96-well plate and cultured under cell culture conditions for 24 hours. After that, the control group and the test substance composition were treated with the cells and further cultured for 24 hours. Real-time PCR was performed to confirm at the gene level, and the test procedure was as follows: Cells treated with the composition of the present invention and salmon PDRN were separated using trypsin and washed with PBS before storing at −80° C. for RNA extraction. For this, at least 1×10⁶ cells per experimental group were processed using the RNeasy Mini Kit (QIAGEN, Cat #74104). The mRNA concentration was measured with NanoDrop (DeNovix, Cat #DS-11), and cDNA was synthesized using RT Master Mix (TOYOBO, Cat #FSQ-201) according to the manufacturer's instructions. Real-time PCR was performed with a reaction mixture containing 10 μL THUNDERBIRD™ Next SYBR qPCR Mix (TOYOBO, Cat #QPS-201), 1 μL (10 μmol/μL) of forward primer, 1 L (10 μmol/μL) of reverse primer, 7 L of Nuclease-Free Water, and 1 L of cDNA. Amplification was carried out using the Rotor-Gene Q 6plex System (Qiagen, Model: Rotor-Gene Q) with 40 cycles of denaturation at 95° C. for 15 seconds, annealing at 62° C. for 1 minute, and extension at 72° C. for 1 minute. Each plate was repeated at least three times. The primers used in the experiment were Qiagen's QuantiTect primer assays (GAPDH; Cat. QT01192646, FLG; Cat. QT00092218, PCOLCE; Cat. QT01005725, CAT; Cat. QT00079674, SOD1; Cat. QT01008651, COX-2; Cat. QT00040586), and the gene expression levels of the samples were quantified with GADPH. The relative expression was calculated using the following formula: R=2^{-[ΔCt sample-ΔCt control]}.

### Experimental Example 5-1: Confirmation of Skin Barrier Enhancement Efficacy by Increased FLG Gene Expression

Filaggrin (FLG) is an essential protein that affects the structure and function of the skin barrier. It is involved in the keratinization process, where keratinocytes proliferate, differentiate into keratinocytes, and form the stratum corneum, maintaining the physical lipid layer structure in the skin barrier. FLG connects keratin intermediate filaments that make up the stratum corneum, maintaining adhesion between keratinocytes. It not only keeps the skin barrier's structure but also acts as a natural moisturizing factor. Therefore, if FLG production decreases, the formation of the keratinocyte membrane decreases, adhesion between keratinocytes weakens, and transepidermal water loss increases, leading to a decrease in the overall robustness of the skin barrier and its ability to prevent moisture loss, which can result in skin dryness and itching. To confirm whether the composition of the present invention has skin barrier enhancement efficacy, changes in FLG gene expression involved in skin barrier improvement were investigated in HaCaT cells (Table 5).

TABLE 5
Comparative relative expression of FLG gene

| | | Relative |
|---|---|---|
| | Treatment | Expression |
| | Concentration | of FLG |
| Treatment Sample | (ppm) | Gene |
| Control Group | — | 1 |
| Positive Control Group | Glyceryl glucoside 1% | 2.20 |
| Centella Asiatica Cell PDRN | 1 | 1.14 |
| Salmon PDRN | | 1.32 |
| Lotus Cell PDRN | | 1.26 |
| Green Tea Cell PDRN | | 1.22 |
| Soybean Cell PDRN | | 1.19 |
| Narcissus Cell PDRN | | 1.28 |
| Pine Cell PDRN | | 1.25 |
| Rice Cell PDRN | | 1.30 |
| Echinacea Cell PDRN | | 1.31 |
| Ginseng Adventitious Root | | 1.42 |
| PDRN | | |

When the composition of the present invention was applied to skin cells at a concentration of 1 ppm, it was observed that there was a tendency to increase FLG gene expression compared to the control group, but no significant difference was confirmed.

### Experimental Example 5-2: Confirmation of Wrinkle Improvement Efficacy by Increased PCOLCE Gene Expression

Collagen exists in various forms within the human body, with collagen type 1 being the most abundant in the skin, playing a crucial role in maintaining skin elasticity and structure. Procollagen C-endopeptidase enhancer (PCOLCE) is a gene that activates enzymes involved in the degradation of procollagen, aiding in the conversion of procollagen to collagen and contributing to collagen production in the skin. To confirm whether the composition of the present invention has wrinkle improvement efficacy, changes in PCOLCE gene expression involved in wrinkle improvement were investigated in fibroblasts (Table 6).

TABLE 6
Comparative Relative Expression of PCOLCE Gene

| | | Relative |
|---|---|---|
| | Treatment | Expression |
| | Concentration | of PCOLCE |
| Treatment Sample | (ppm) | Gene |
| Control Group | — | 1.00 |
| Positive Control Group | TGF-β1 5 ng/mL | 1.45 |
| Centella Asiatica Cell PDRN | 1 | 1.52 |
| Salmon PDRN | — | 1.91 |
| Lotus Cell PDRN | — | 1.38 |
| Green Tea Cell PDRN | — | 1.25 |
| Soybean Cell PDRN | — | 1.37 |
| Narcissus Cell PDRN | — | 1.30 |
| Pine Cell PDRN | — | 1.40 |
| Rice Cell PDRN | — | 1.37 |
| Echinacea Cell PDRN | — | 1.41 |
| Ginseng Adventitious Root | — | 1.57 |
| PDRN | | |

When the composition of the present invention was treated to skin cells at a concentration of 1 ppm, the expression of the PCOLCE gene was increased compared to the control group, confirming a wrinkle improvement effect.

### Experimental Example 5-3: Confirmation of Antioxidant Efficacy by Increased Expression of CAT and SOD1 Genes

CAT (Catalase) and SOD1 (Superoxide Dismutase 1) are enzymes that play important roles in the body's antioxidant defense system. CAT breaks down hydrogen peroxide, a reactive oxygen species, into water and oxygen, thereby reducing oxidative stress within cells and protecting cells from oxidative damage. SOD1 is an enzyme that converts superoxide anions into hydrogen peroxide, effectively removing reactive oxygen species generated within cells and preventing oxidative damage. To confirm whether the composition of the present invention has antioxidant efficacy, changes in the expression levels of CAT and SOD1 genes involved in antioxidant activity were investigated in cells (Tables 7 and 8).

TABLE 7
Comparative Relative Expression of CAT Gene

| | | Relative |
|---|---|---|
| | Treatment | Expression |
| | Concentration | of CAT |
| Treatment Sample | (ppm) | Gene |
| Control Group | — | 1 |
| Centella Asiatica Cell PDRN | 1 | 2.5 |
| Salmon PDRN | | 1.8 |
| Lotus Cell PDRN | | 1.9 |
| Green Tea Cell PDRN | | 2.1 |
| Soybean Cell PDRN | | 1.5 |
| Narcissus Cell PDRN | | 2.1 |
| Pine Cell PDRN | | 1.9 |
| Rice Cell PDRN | | 2.0 |
| Echinacea Cell PDRN | | 1.8 |
| Ginseng Adventitious Root | — | 2.2 |
| PDRN | | |

TABLE 8
Comparative Relative Expression of SOD1 Gene

| | | Relative |
|---|---|---|
| | Treatment | Expression |
| | Concentration | of SOD1 |
| Treatment Sample | (ppm) | Gene |
| Control Group | — | 1 |
| Centella Asiatica Cell PDRN | 1 | 1.4 |
| Salmon PDRN | | 1.3 |
| Lotus Cell PDRN | | 1.2 |
| Green Tea Cell PDRN | | 1.3 |
| Soybean Cell PDRN | | 1.3 |
| Narcissus Cell PDRN | | 1.2 |
| Pine Cell PDRN | | 1.4 |
| Rice Cell PDRN | | 1.3 |
| Echinacea Cell PDRN | | 1.4 |
| Ginseng Adventitious Root | | 1.4 |
| PDRN | | |

When the composition of the present invention was applied to skin cells at a concentration of 1 ppm, it was confirmed that the expression of CAT and SOD1 genes increased compared to the control group, indicating antioxidant effects. For CAT, it was confirmed that the effect was significantly superior to that of the commercially available salmon PDRN.

### Experimental Example 5-4: Confirmation of Anti-Inflammatory Efficacy by Increased COX-2 Gene Expression

COX2 (Cyclooxygenase-2) is an enzyme related to inflammatory responses, promoting the production of prostaglandins within cells and inducing inflammation and pain responses. COX2 expression increases particularly in response to acute inflammatory reactions or external stimuli and is known as an important factor related to inflammatory diseases. To confirm whether the composition of the present invention has anti-inflammatory efficacy, changes in COX2 gene expression involved in inflammatory responses were investigated in fibroblasts (Table 9).

TABLE 9
Comparative Relative Expression of COX-2 Gene

| | | Relative |
|---|---|---|
| | Treatment | Expression |
| | Concentration | of COX-2 |
| Treatment Sample | (ppm) | Gene |
| Control Group | — | 1 |
| Positive Control Group | Dexamethasone 5 μM | 0.25 |
| Centella Asiatica Cell PDRN | 1 | 0.70 |
| Salmon PDRN | | 0.64 |
| Lotus Cell PDRN | | 0.55 |
| Green Tea Cell PDRN | | 0.42 |
| Soybean Cell PDRN | | 0.69 |
| Narcissus Cell PDRN | | 0.32 |
| Pine Cell PDRN | | 0.51 |
| Rice Cell PDRN | | 0.69 |
| Echinacea Cell PDRN | | 0.71 |
| Ginseng Adventitious Root | | 0.50 |
| PDRN | | |

When the composition of the present invention was applied to skin cells at a concentration of 1 ppm, it was confirmed that the expression of the COX2 gene was significantly reduced compared to the control group, indicating that the composition of the present invention has anti-inflammatory effects.

### Experimental Example 6: Evaluation of Skin Barrier Efficacy by Quantification of Involucrin and Claudin 1 Protein Using Immunostaining

Immunofluorescence staining began by thoroughly washing the cells treated with the composition of the present invention and salmon PDRN with phosphate buffer. The cells were then fixed with 4% fixative at room temperature for 1 hour and permeabilized with 1% Triton X-100 at 37° C. for 1 hour. After that, the cells were washed four times with phosphate buffer. The cells were incubated in 2% bovine serum albumin solution to minimize non-specific binding for 2 hours. Subsequently, the cells were treated with primary antibodies Involucrin (1:200, PA5-32454, ThermoFisher Scientific) and Claudin 1 (1:200, 51-9000, Thermo Fisher Scientific) and incubated overnight at 4° C. Then, the cells were washed several times with phosphate buffer and treated with the secondary antibody FITC-conjugated anti-rabbit polyclonal antibody (1:200; ab6717; Abcam) at 37° C. for 2 hours in the dark. After the secondary antibody reaction, the cells were washed with phosphate buffer and counterstained with 5 g/mL Hoechst-33342 for 8 minutes. Finally, the cells were thoroughly washed, mounted on slides, covered with a coverslip, and observed under a fluorescence microscope.

### Experimental Example 6-1: Confirmation of Skin Barrier Efficacy by Involucrin Protein Expression

Involucrin is a protein that plays an important role in the keratinization process of the epidermis, contributing to the formation of the skin barrier and protecting the skin from external stimuli. Involucrin expression increases during the differentiation process of keratinocytes and is essential for enhancing the strength and stability of the skin barrier. To confirm whether the composition of the present invention has skin barrier enhancement efficacy, changes in involucrin gene expression involved in skin protection were investigated in fibroblasts (Table 10).

TABLE 10
Comparative Relative Expression of Involucrin Protein

| | | Relative |
|---|---|---|
| | Treatment | Expression |
| | Concentration | of Involucrin |
| Treatment Sample | (ppm) | Protein |
| Control Group | — | 1 |
| Composition of the | 1 | 1.3 |
| Present Invention | | |
| Salmon PDRN | 1 | 1.4 |

When the composition of the present invention was applied to skin cells at a concentration of 1 ppm, it was confirmed that the expression of Involucrin protein significantly increased compared to the control group, indicating skin barrier effects.

### Experimental Example 6-2: Confirmation of Skin Barrier Efficacy by Claudin 1 Protein Expression

Claudin 1 is one of the proteins that form tight junctions between skin cells, playing an important role in maintaining skin moisture and blocking the penetration of external substances. Claudin 1 strengthens cell-to-cell adhesion, protecting the skin barrier and defending the skin from external environments. To confirm whether the composition of the present invention has skin barrier enhancement efficacy, changes in Claudin 1 gene expression involved in skin barrier formation were investigated in fibroblasts (Table 11)

TABLE 11
Comparative Relative Expression of Claudin 1 Protein

| | Treatment | Relative Expression |
|---|---|---|
| Treatment | Concentration | of Claudin 1 |
| Sample | (ppm) | Protein |
| Control Group | — | 1 |
| Composition of the | 1 | 1.6 |
| Present Invention | | |
| Salmon PDRN | 1 | 1.7 |

When the composition of the present invention was applied to skin cells at a concentration of 1 ppm, it was confirmed that the expression of Claudin 1 protein significantly increased compared to the control group, indicating skin barrier effects.

### Experimental Example 7: Measurement of ATP and Fatty Acid (FA) Expression Using BODIPY Detection Reagent

The BODIPY reagent used for lipid staining is a fluorescent dye that selectively stains neutral lipids and lipid droplets within cells, visualizing the state of fatty acid accumulation. BODIPY is lipophilic and binds to intracellular fatty acids, emitting fluorescence, making it suitable for confirming the amount of lipid accumulation within cells. In this experiment, the BODIPY reagent was used to confirm the accumulation of ATP and fatty acids.

### Experimental Example 7-1: Measurement of Intracellular ATP Content Using BODIPY Detection Reagent

Measuring ATP production is essential for evaluating cells' energy metabolism state and physiological function. ATP is a vital energy source for cell activity and survival and plays a crucial role in the fatty acid oxidation. Therefore, evaluating ATP production can help confirm the effect of the composition on intracellular energy metabolism and identify metabolic changes related to fatty acid accumulation. In this experiment, the BODIPY reagent was used to visualize fatty acid accumulation and measure ATP production to evaluate the metabolic state of the cells (Table 12).

TABLE 12
Comparative Relative Intracellular ATP Expression

| | Concentration | Relative Intracellular ATP |
|---|---|---|
| Sample | (ppm) | Expression |
| Control Group | — | 1 |
| Composition of the Present | 1 | 1.7 |
| Invention | | |
| Salmon PDRN | 1 | 1.7 |

When the composition of the present invention was applied to skin cells at a concentration of 1 ppm, intracellular ATP expression significantly increased compared to the control group, confirming that the composition of the present invention enhances intracellular energy metabolism and has similar efficacy to the commercial product, salmon PDRN.

### Experimental Example 7-2: Measurement of Intracellular FA Content Using BODIPY Detection Reagent

Confirming fatty acid accumulation plays a vital role in evaluating changes in intracellular energy and lipid metabolism. Fatty acids are used as an energy source or stored as lipid droplets and are closely related to cell metabolic balance. By measuring the amount of fatty acid accumulation, it is possible to evaluate the effect of a specific composition on intracellular lipid metabolism and to determine what changes it induces in energy metabolism and fatty acid oxidation processes. The purpose of this experiment was to comprehensively evaluate the effects of the composition on cell metabolism and lipid accumulation by confirming fatty acid accumulation and ATP production using the BODIPY reagent (Table 13).

TABLE 13
Comparative Relative Intracellular FA Expression

| | Concentration | Relative Intracellular FA |
|---|---|---|
| Sample | (ppm) | Expression |
| Control Group | — | 1 |
| Composition of the Present | 1 | 1.4 |
| Invention | | |
| Salmon PDRN | 1 | 1.5 |

When the composition of the present invention was applied to skin cells at a concentration of 1 ppm, intracellular FA expression significantly increased compared to the control group, confirming that the composition of the present invention enhances intracellular fatty acid accumulation and has similar efficacy to the commercial product, salmon PDRN.

### Experimental Example 8: Evaluation of Wrinkle Improvement, Elasticity, Skin Barrier Enhancement, and Moisturizing Efficacy in Reconstructed Skin Tissue

The composition of the present invention was applied to reconstructed skin tissue every 48 hours. After 5 days, the tissue was collected and fixed in 10% neutral buffered formalin for 24 hours. The tissue was embedded in OCT compound, sectioned into 12 m slices, attached to gelatin-coated slides, dried, and stored for staining. The prepared tissue sections on the slides were hydrated in distilled water, stained with hematoxylin solution at room temperature for 2 minutes for nuclear staining, washed, and stained with eosin Y solution for 3 minutes. The sections were dehydrated, cleared with alcohol and xylene, mounted with Canada balsam, and observed under a microscope (Leica THUNDER Imager Tissue microscope).

The prepared tissue sections on the slides were hydrated in distilled water for immunofluorescence staining. The sections were boiled in 0.01M citrate solution (pH 6.0) using a microwave for 1 minute, then left to stand at room temperature for 20 minutes. This was blocked using anti-goat serum, and the sections were incubated overnight at 4° C. with primary antibodies Aquaporin-3 (1:200), Filaggrin (1:100) (Santacruz), and Collagen Type I (1:500) (Invitrogen). After washing with PBS, the sections were incubated for 1 hour at room temperature with secondary antibodies Goat anti-mouse IgG Alexa Fluor 555 (1:500) and Goat anti-rabbit IgG Alexa Fluor 488 (1:500) (abcam), washed with PBS, and mounted with mounting medium containing DAPI.

The sections were observed under a fluorescence microscope (Leica THUNDER Imager Tissue microscope). Images taken at 200× magnification with the fluorescence microscope were quantified using the Image J program to analyze the positive staining area objectively. The positive area (%) is the ratio of positive area and total area; the Change Rate (%) is the ratio of [test group positive area-untreated group positive area] and [untreated group positive rate]

### Experimental Example 8-1: General Morphology in Reconstructed Skin Tissue

To evaluate the composition of the present invention, the general morphology of the reconstructed skin tissue was examined. The stratum corneum was thick and slightly layered, and the epidermis showed 5-6 cell layers with good morphology. The dermis showed well-defined collagen fibers around fibroblasts, confirming that it is suitable for evaluating wrinkle improvement, elasticity, skin barrier enhancement, and moisturizing effects.

### Experimental Example 8-2: Analysis of Proteins Involved in Skin Barrier

Filaggrin (FLG) is a protein that plays an essential role in forming the stratum corneum in the epidermis. An increase in FLG indicates the formation of a normal skin barrier, preventing the evaporation of skin moisture. FLG was stained with green fluorescence from the granular layer to the stratum corneum of the epidermis. The blue fluorescence stained the cell nuclei.

The test group using the composition of the present invention showed a significant change in FLG expression compared to the control group, with a change rate of 82.62%. This indicates that the composition of the present invention can help form the skin barrier by increasing FLG expression and enhance skin moisturization by increasing natural moisturizing factors (Table 14).

TABLE 14
Statistical Analysis of Filaggrin Positive
Staining Area Comparison Results (N = 3)

| | | | Change Rate | |
|---|---|---|---|---|
| | | | (%) | ²p-value |
| | ¹Positive Area | Standard | Vs. Control | Vs. Control |
| Group | (%) Mean | Deviation | Group | Group |
| Control Group | 19.16 | 2.35 | — | — |
| Composition of the Present | 34.99 | 1.24 | 82.62 ↑ | 0.000007 |
| Invention | | | | |
| Salmon-Derived PDRN | 31.67 | 1.00 | 65.28 ↑ | 0.000020 |

¹An increase in the mean value indicates an increase in Filaggrin;
²If p < 0.05, it indicates a significant difference;
3: * p < 0.05, ** p < 0.01, *** p < 0.001 indicate significance levels, with *** representing the highest significance and thus the highest efficacy.

Experimental Example 8-3: Analysis of Proteins Involved in Skin Moisturization

Aquaporin 3 (AQP3), primarily expressed in the epidermal layer of the skin, is closely related to skin moisturization. Unlike the epidermal layer, it is not expressed in the stratum corneum. AQP3 was stained with red fluorescence in the basal and spinous layers, while the blue fluorescence stained the cell nuclei.

As a result of the analysis, the test group using the composition of the present invention showed a significant change in AQP3 expression compared to the control group, with a change rate of 45.93%. This indicates that the composition of the present invention can help improve skin moisturization by increasing AQP3 expression, thereby regulating water movement between epidermal cells (Table 15).

TABLE 15
Statistical Analysis of Aquaporin 3 Positive Staining Area Comparison Results (N = 3)

| | ¹Positive Area | Standard | Change Rate (%) | ²p-value |
|---|---|---|---|---|
| Group | (%) Mean | Deviation | Vs. Control Group | Vs. Control Group |
| Control Group | 25.82 | 1.60 | — | — |
| Composition of the | 37.68 | 2.86 | 45.93 ↑ | 0.000115 |
| Present Invention | | | | |
| Salmon-Derived PDRN | 30.99 | 3.63 | 20.01 ↑ | 0.018740 |

¹An increase in the mean value indicates an increase in Filaggrin;
²If p < 0.05, it indicates a significant difference;
³ * p < 0.05, ** p < 0.01, *** p < 0.001 indicate significance levels, with *** representing the highest significance and thus the highest efficacy.

### Experimental Example 8-4: Analysis of Proteins Involved in Skin Wrinkle Improvement

Collagen Type I (COL I) constitutes 70% of the dermis and is essential in preventing skin wrinkles. An increase in COL I indicates the potential to avoid wrinkle formation. COL I was stained with green fluorescence in the dermal layer, while the blue fluorescence stained the cell nuclei.

As a result of the analysis, the test group using the composition of the present invention showed a significant change in COL I expression compared to the control group, with a change rate of 35.53%. This indicates that the composition of the present invention can help improve skin wrinkles by increasing COL I expression (Table 16).

TABLE 16
Statistical Analysis of Collagen Type I Positive Staining Area Comparison Results (N = 3)

| | ¹Positive Area | Standard | Change Rate (%) | ²p-value |
|---|---|---|---|---|
| Group | (%) Mean | Deviation | Vs. Control Group | Vs. Control Group |
| Control Group | 15.14 | — | — | — |
| Composition of the | 20.52 | 2.49 | 35.53 ↑ | 0.009890 |
| Present Invention | | | | |
| Salmon-Derived PDRN | 24.05 | 1.57 | 58.82 ↑ | 0.000325 |

¹An increase in the mean value indicates an increase in Filaggrin;
²If p < 0.05, it indicates a significant difference;
³ * p < 0.05, ** p < 0.01, *** p < 0.001 indicate significance levels, with *** representing the highest significance and thus the highest efficacy.

Through testing the composition of the present invention on reconstructed skin tissue, it was confirmed that the expression of proteins involved in skin wrinkle improvement (Collagen Type I), skin barrier, and moisturization (Aquaporin 3, Filaggrin) increased, indicating that it can help improve elasticity, strengthen the skin barrier, and enhance moisturization.

### Experimental Example 9: Efficacy Evaluation in Human Clinical Trials (Improvement of 6 Types of Wrinkles, Skin Elasticity, Skin Density, and Facial Cheek Lifting)

### Experimental Example 9-1: Evaluation of 6 Types of Wrinkles (Forehead, Glabella, Crow's Feet, Nasolabial Folds, Perioral Wrinkles, Neck) Using Antera 3D® CS

To evaluate the improvement of 6 types of wrinkles (forehead, Glabella, crow's feet, nasolabial folds, perioral wrinkles, neck), the bilateral facial and neck areas were divided into a test group containing the composition of the present invention and a control group without the composition. Using the Wrinkles small mode (1.00 mm filter) of Antera 3D® CS, the subjects' forehead, Glabella, crow's feet, and neck areas were measured and analyzed before use and after 4 weeks of use. The nasolabial folds and perioral regions were measured and analyzed using the Wrinkles medium mode (2.00 mm filter) before and after 4 weeks of use.

The analysis was conducted by setting circles of 33.1 mm for the forehead wrinkles, 11.7 mm for the Glabella wrinkles, 12.8 mm for the crow's feet, and 30.8 mm for the neck wrinkles. Polygonal shapes were used to analyze the nasolabial folds and perioral wrinkles. The analysis values used the Indentation Index (A.U.), representing the selected area's wrinkle index.

Suppose the analysis values of the test group containing the composition of the present invention significantly decreased after 4 weeks of use compared to before use, and the change rate of the test group was significantly higher than that of the control group. In that case, it indicates an improvement in the 6 types of wrinkles (forehead, Glabella, crow's feet, nasolabial folds, perioral wrinkles, neck) (Table 17).

The results of evaluating the 6 types of wrinkles (forehead, Glabella, crow's feet, nasolabial folds, perioral wrinkles, neck) using Antera 3D® CS are as follows.

TABLE 17
Forehead Wrinkle Measurement Results (n = 20)

| | | | p-value | | |
|---|---|---|---|---|---|
| | | Mean ± | | Between | Change |
| | | Standard | Within Group | Group | Rate |
| Measurement Time Point | | Deviation | Comparison* | Comparison† | (%) |
| Test Group | Before Use | 8.49 ± 1.36 | <0.001 | <0.001 | 11.23 |
| | After 4 Weeks of Use | 7.51 ± 1.06 | | | |
| Control Group | Before Use | 8.26 ± 1.41 | <0.001 | | 5.40 |
| | After 4 Weeks of Use | 7.81 ± 1.36 | | | |

*Significantly different at p < 0.05 compared between time points by paired t-test;
†Significantly different at p < 0.05 compared between groups by Independent two sample t-test

Compared to before use, the Indentation Index value representing the forehead wrinkle index significantly decreased by 11.23% in the test group and by 5.4% in the control group after 4 weeks of use (p<0.05) (Table 18).

TABLE 18
Glabella Wrinkle Measurement Results (n = 20)

| | | | p-value | | |
|---|---|---|---|---|---|
| | | Mean ± | | Between | Change |
| | | Standard | Within Group | Group | Rate |
| Measurement Time Point | | Deviation | Comparison* | Comparison† | (%) |
| Test Group | Before Use | 10.50 ± 1.53 | <0.001 | 0.037 | 7.98 |
| | After 4 Weeks of Use | 9.62 ± 1.33 | | | |
| Control Group | Before Use | 10.21 ± 1.65 | <0.001 | | 3.94 |
| | After 4 Weeks of Use | 9.79 ± 1.59 | | | |

*Significantly different at p < 0.05 compared between time points by paired t-test
†Significantly different at p < 0.05 compared between groups by Mann-Whitney U test

Compared to before use, the Indentation Index value representing the Glabella wrinkle index significantly decreased by 7.98% in the test group and by 3.94% in the control group after 4 weeks of use (p<0.05) (Table 19).

TABLE 19
Crow's Feet Wrinkle Measurement Results (n = 20)

| | | | p-value | | |
|---|---|---|---|---|---|
| | | Mean ± | | Between | Change |
| | | Standard | Within Group | Group | Rate |
| Measurement Time Point | | Deviation | Comparison* | Comparison† | (%) |
| Test Group | Before Use | 10.14 ± 1.93 | <0.001 | 0.035 | 8.53 |
| | After 4 Weeks of Use | 9.23 ± 1.63 | | | |
| Control Group | Before Use | 9.98 ± 2.42 | 0.003^{w} | | 3.80 |
| | After 4 Weeks of Use | 9.60 ± 2.43 | | | |

*Significantly different at p < 0.05 compared between time points by paired t-test;
†Significantly different at p < 0.05 compared between time points;
^{w}Wilcoxon signed rank test;
†Significantly different at p < 0.05 compared between time points

Compared to before use, the Indentation Index value representing the crow's feet wrinkle index significantly decreased by 8.53% in the test group and by 3.80% in the control group after 4 weeks of use (p<0.05) (Table 20).

TABLE 20
Nasolabial Fold Wrinkle Measurement Results (n = 20)

| | | | p-value | | |
|---|---|---|---|---|---|
| | | Mean ± | | Between | Change |
| | | Standard | Within Group | Group | Rate |
| Measurement Time Point | | Deviation | Comparison* | Comparison† | (%) |
| Test Group | Before Use | 25.71 ± 4.96 | <0.001 | <0.001 | 13.42 |
| | After 4 Weeks of Use | 22.02 ± 3.95 | | | |
| Control Group | Before Use | 25.32 ± 5.56 | 0.003 | | 3.66 |
| | After 4 Weeks of Use | 24.29 ± 4.87 | | | |

*Significantly different at p < 0.05 compared between time points by paired t-test;
†Significantly different at p < 0.05 compared between groups by Mann-Whitney U test;
Compared to before use, the Indentation Index value representing the nasolabial fold wrinkle index significantly decreased by 13.42% in the test group and by 3.66% in the control group after 4 weeks of use (p < 0.05) (Table 21).

TABLE 21
Effect of measuring wrinkles around the mouth (n = 20)

| | | | p-value | | |
|---|---|---|---|---|---|
| | | Mean ± | | Between | Change |
| | | Standard | Within Group | Group | Rate |
| Measurement Time Point | | Deviation | Comparison* | Comparison† | (%) |
| Test Group | Before Use | 30.63 ± 6.36 | <0.001 | 0.001 | 15.67 |
| | After 4 Weeks of Use | 25.63 ± 5.25 | | | |
| Control Group | Before Use | 31.51 ± 8.59 | 0.008 | | 5.78 |
| | After 4 Weeks of Use | 29.57 ± 8.08 | | | |

*Significantly different at p < 0.05 compared between time points by paired t-test;
†Significantly different at p < 0.05 compared between groups by Mann-Whitney U test

Compared to before use, the Indentation Index value, which indicates the wrinkle index around the mouth, significantly decreased after 4 weeks of use: by 15.67% in the test group and by 5.78% in the control group (p<0.05) (Table 22).

TABLE 22
Effect of measuring neck wrinkles (n = 20)

| | | | p-value | | |
|---|---|---|---|---|---|
| | | Mean ± | | Between | Change |
| | | Standard | Within Group | Group | Rate |
| Measurement Time Point | | Deviation | Comparison* | Comparison† | (%) |
| Test Group | Before Use | 10.40 ± 1.93 | <0.001 | 0.001 | 11.36 |
| | After 4 Weeks of Use | 9.16 ± 1.45 | | | |
| Control Group | Before Use | 10.03 ± 1.70 | <0.001 | | 6.29 |
| | After 4 Weeks of Use | 9.39 ± 1.61 | | | |

*Significantly different at p < 0.05 compared between time points by paired t-tes;
†Significantly different at p < 0.05 compared between groups by Independent two-sample t-test

Compared to before use, the Indentation Index value, which indicates the wrinkle index around the neck, significantly decreased after 4 weeks of use by 11.36% in the test group and by 6.29% in the control group (p<0.05).

The comparison between groups showed that the rate of change in the test group containing the composition of the present invention was statistically significantly higher than that of the control group in the forehead, Glabella, eye area, nasolabial folds, mouth area, and neck. This confirmed the improvement of six types of wrinkles (forehead, Glabella, eye area, nasolabial folds, mouth area, and neck).

### Experimental Example 9-2: Evaluation of Skin Elasticity Improvement Using Cutometer® Dual MPA 580

To evaluate the improvement in skin elasticity, the cheeks were divided into a test group containing the composition of the present invention and a control group without the composition. The cheeks of the study subjects were measured before use and after 4 weeks of use using the Cutometer® dual MPA 580. The measurement values used were the R2(%) values, which indicate skin elasticity. The analysis values of the test group containing the invention's composition showed a statistically significant increase after 4 weeks of use compared to before use. When the rate of change in the test group was statistically significantly higher than that of the control group, it indicated an improvement in skin elasticity (Table 23).

TABLE 23
Results of skin elasticity improvement measurement (n = 20)

| | | | p-value | | |
|---|---|---|---|---|---|
| | | Mean ± | | Between | Change |
| | | Standard | Within Group | Group† | Rate |
| Measurement Time Point | | Deviation | Comparison* | Comparison | (%) |
| Test Group | Before Use | 0.604 ± 0.019 | <0.001 | <0.001 | 4.09 |
| | After 4 Weeks of Use | 0.628 ± 0.018 | | | |
| Control Group | Before Use | 0.606 ± 0.023 | <0.001 | | 1.81 |
| | After 4 Weeks of Use | 0.617 ± 0.022 | | | |

*Significantly different at p < 0.05 compared between time points by paired t-test;
†Significantly different at p < 0.05 compared between groups by Independent two-sample t-test

Compared to before use, the R2 value, which indicates skin elasticity, significantly increased after 4 weeks of use: 4.09% in the test group and 1.81% in the control group (p<0.05). The comparison between groups showed that the rate of change in the test group was statistically significantly higher than that of the control group, indicating an improvement in skin elasticity (p<0.05).

### Experimental Example 9-3: Evaluation of Skin Density Improvement Using DUB® SkinScanner

To evaluate the improvement in skin density, the eye areas were divided into a test group containing the composition of the present invention and a control group without the composition. The eye areas of the study subjects were measured and analyzed before use and after 4 weeks of use using the DUB® SkinScanner. The analysis values used were the Dermis Density (%) values, which indicate skin density. The analysis values of the test group containing the invention's composition showed a statistically significant increase after 4 weeks of use compared to before use. When the rate of change in the test group was statistically significantly higher than that of the control group, it indicated an improvement in skin density (Table 24).

TABLE 24
Results of skin density improvement measurement (n = 20)

| | | | p-value | | |
|---|---|---|---|---|---|
| | | Mean ± | | Between | Change |
| | | Standard | Within Group | Group | Rate |
| Measurement Time Point | | Deviation | Comparison* | Comparison† | (%) |
| Test Group | Before Use | 0.604 ± 0.019 | <0.001 | <0.001 | 8.30 |
| | After 4 Weeks of Use | 0.628 ± 0.018 | | | |
| Control Group | Before Use | 0.606 ± 0.023 | <0.001 | | 2.57 |
| | After 4 Weeks of Use | 0.617 ± 0.022 | | | |

*Significantly different at p < 0.05 compared between time points by paired t-test;
†Significantly different at p < 0.05 compared between groups by Independent two sample t-test

Compared to before use, the Dermis Density value, which indicates skin density, significantly increased after 4 weeks of use: by 8.30% in the test group and by 2.57% in the control group (p<0.05). The comparison between groups showed that the rate of change in the test group was statistically significantly higher than that of the control group, indicating an improvement in skin density (p<0.05).

### Experimental Example 9-4: Evaluation of Facial Cheek Lifting Effect Using F-ray and Image-pro® 10

To evaluate the facial cheek-lifting effect, the facial areas were divided into a test group containing the composition of the present invention and a control group without the composition. The facial areas of the study subjects were photographed before and after 4 weeks of use using the F-ray and analyzed using the image analysis program Image-pro® 10. The analysis values used were the angle (°) values, which indicate the degree of skin sagging by drawing a horizontal line from the tip of the nose and a tangent to the contour line derived from the nasolabial folds. The analysis values of the test group containing the invention's composition showed a statistically significant decrease after 4 weeks of use compared to before use. When the rate of change in the test group was statistically significantly higher than that of the control group, it indicated a facial cheek-lifting effect (Table 25).

TABLE 25
Results of facial cheek lifting measurement (n = 20)

| | | | p-value | | |
|---|---|---|---|---|---|
| | | Mean ± | | Between | Change |
| | | Standard | Within Group | Group | Rate |
| Measurement Time Point | | Deviation | Comparison* | Comparison† | (%) |
| Test Group | Before Use | 41.99 ± 4.82 | <0.001 | <0.001 | 3.67 |
| | After 4 Weeks of Use | 40.44 ± 4.59 | | | |
| Control Group | Before Use | 41.68 ± 4.85 | <0.001 | | 1.26 |
| | After 4 Weeks of Use | 41.16 ± 4.82 | | | |

*Significantly different at p < 0.05 compared between time points by paired t-test
†Significantly different at p < 0.05 compared between groups by Mann-Whitney U test

Compared to before use, the angle value, which indicates the degree of skin sagging, significantly decreased after 4 weeks of use by 3.67% in the test group and 1.26% in the control group (p<0.05). The comparison between groups showed that the rate of change in the test group was statistically significantly higher than that of the control group, indicating a facial cheek-lifting effect (p<0.05).

In one embodiment, this invention is a method of compounding a polydeoxyribonucleotide (PDRN) mixture ranging in molecular weights from 50 to 1500 kDa, inclusive, derived from plant cells secured from comprising:
a) Washing the leaves to remove dirt and contaminants;
b) Freezing the material in liquid nitrogen to prevent enzymatic degradation
c) Grinding the frozen material into a fine powder;
d) Extracting DNA in a DNA extraction buffer to release nucleic acids;
e) Incubating the buffer with a proteinase K to digest proteins;
f) Precipitating protein to separate nucleic acids using cold ethanol or isopropanol;
g) Removing impurities by washing with ethanol and dissolving in nuclease-free water or a buffer;
h) Mechanically shearing by sonification or mechanical stress or using DNase I or other nucleases to reduce DNA to a smaller size;
i) Ultrafiltration or column-based purification;
j) Analyzing by using gel electrophoresis and UV spectrophotometry to ensure particular size and absence of impurities;
k) Formulating into a topical formulation such as a gel, cream, or hydrogel to stabilize the DNA.

In another embodiment, the present invention is a method of the PDRN preparation comprising following steps:
a) Immersing leaves of Centella asiatica plant in 70% ethanol for 30 seconds, followed by washing with water;
b) Disinfecting the leaves with a solution comprising 30% bleach and Tween 20 for 20 minutes, followed by washing the leaves with water three times;
c) Scratching the leaves with a knife and placing them in a culture medium containing 1 mg/mL zeatin, 3% sucrose, and 8 g/L agar at 25° C. in the dark to induce cell formation;
d) Culturing the induced plant cells in a bioreactor at 25° C. providing an air supply of 0.1 vvm for five weeks;
e) Harvesting the culture by filtering them on a cloth filter paper of 0.22 to 0.25-micron pores size;
f) Immersing the filter paper in 95% ethanol for 1 to 2 hours to decolorize the cells and freezing the cells at −20 to −80° C.;
g) Disrupting cells by thawing the cells, followed by immersing the thawed cells in Lysis Buffer (0.5 M sodium chloride, Disodium Cocoyl Glutamate) at 65° C. for 60 minutes;
h) Exposing the cells for 30 minutes to 1 hour to a radio frequency of 150 to 200 kw;
i) Removing proteins by adding endo- and exo-proteases to react at room temperature for 30-60 minutes;
j) Removing impurities by filtering and centrifugation and collecting supernatant;
k) Diluting the supernatant by 50% with 95% ethanol and precipitating it at −20 to −80° C. for 12 hours;
l) Centrifuging the precipitated supernatant to collect DNA pellets;
m) Washing the DNA pellets and further collecting DNA pellets by centrifugation;
n) Drying the DNA pellets at 40 to 65° C. for 6 to 12 hours;
o) Dissolving the DNA pellets in water;
p) Treating DNA pellet solution with ultrasonic radiation (28 kHz) for 90-120 minutes to separate low molecular weight DNA (PDRN) of 100-200 bp;
q) Certifying purity (A260/A280=1.8-2.0);
r) Storing PDRN after passing it through a 0.45 m to 0.22 m filter stage.

In another embodiment, the present invention is a method of compounding a polydeoxyribonucleotide (PDRN) that involves using plant cells sourced from Centella asiatica, Triticum aestivum, Hordeum vulgare, Zea mays, Glycine max, Lens culinaris, Oryza sativa, Spirulina vulgaris, Chlorella vulgaris, Panax ginseng, Aloe vera, Spinacia oleracea, Lactuca sativa, Nicotiana tabacum, Solanum tuberosum, Spirulina, Chlorella, Brassica oleracea, and Rosa, or a combination thereof.

In another embodiment, the invention is:
a) Employed as an active ingredient for a cosmetic composition, a pharmaceutical composition, or a quasi-formulation composition.
b) Used in a cosmetic formulation to protect skin from ultraviolet rays, strengthen the skin barrier, moisturize, improve wrinkles, suppress skin stress, regenerate skin, heal wounds, reduce skin aging, or a combination thereof.
c) Formulated in a pharmaceutical composition for the prevention or treatment of any skin disease caused by exposure to ultraviolet rays, psoriasis, skin wounds, scars, or skin inflammation.
d) Formulated as a quasi-drug composition for improving skin by protecting skin from ultraviolet rays, strengthening the skin barrier, moisturizing, improving wrinkles, suppressing skin stress, regenerating skin, healing wounds, and reducing aging.

1. A composition of polydeoxyribonucleotide (PDRN) mixture ranging in molecular weights from 50 to 1500 kDa, inclusive, extracted from plant cells comprising leaves, stems or flowers or Centella asiatica, Triticum aestivum, Hordeum vulgare, Zea mays, Glycine max, Lens culinaris, Oryza sativa, Spirulina vulgaris, Chlorella vulgaris, Panax ginseng, Aloe vera, Spinacia oleracea, Lactuca sativa, Nicotiana tabacum, Solanum tuberosum, Spirulina, Chlorella, Brassica oleracea, and Rosa, or a combination thereof.

2. The composition of polydeoxyribonucleotide (PDRN) mixture of claim 1, wherein the PDRN mixture is employed as an active ingredient for a cosmetic composition, a pharmaceutical composition, a quasi-formulation composition, or a combination thereof.

3. The composition of polydeoxyribonucleotide (PDRN) mixture of claim 2, wherein the said compositions are in a topical application form comprising a gel, cream, and hydrogel, or a combination thereof.

4. The composition of polydeoxyribonucleotide (PDRN) mixture of claim 3, wherein the cosmetic composition, the pharmaceutical composition, or the quasi-formulation composition is used to protect skin from ultraviolet rays, strengthen the skin barrier against environmental chemicals, moisturize skin, reduce skin wrinkles, reduce skin stress, regenerate skin, heal skin wounds, and reduce skin aging, or a combination thereof.

5. The composition of polydeoxyribonucleotide (PDRN) mixture of claim 2, wherein the pharmaceutical composition is used for the prevention or treatment of any skin disease caused by exposure to ultraviolet rays, psoriasis, skin wounds, skin scars, skin inflammation, or a combination thereof.

6. The composition of polydeoxyribonucleotide (PDRN) mixture of claim 1, wherein the plant cells are isolated using a series of steps comprising:
a. Immersing leaves of Centella asiatica plant in 70% ethanol for 30 seconds, followed by washing with water;
b. Disinfecting the leaves with a solution comprising 30% bleach and Tween 20 for 20 minutes, followed by washing the leaves with water three times;
c. Scratching the leaves with a knife and placing them in a culture medium containing 1 mg/mL zeatin, 3% sucrose, and 8 g/L agar at 25° C. in the dark to induce cell formation;
d. Culturing the induced plant cells in a bioreactor at 25° C. providing an air supply of 0.1 vvm for five weeks;
e. Harvesting the culture by filtering them on a cloth filter paper of 0.22 to 0.25-micron pores size;
f. Immersing the filter paper in 95% ethanol for 1 to 2 hours to decolorize the cells and freezing the cells at −20 to −80° C.

7. The composition of polydeoxyribonucleotide (PDRN) mixture of claim 6, wherein the polydeoxyribonucleotide (PDRN) mixture is produced in a series of steps comprising:
a. Disrupting cells by thawing the cells, followed by immersing the thawed cells in Lysis Buffer (0.5 M sodium chloride, Disodium Cocoyl Glutamate) at 65° C. for 60 minutes;
b. Exposing the cells for 30 minutes to 1 hour to radio frequency of 150 to 200 kw;
c. Removing proteins by adding endo- and exo-proteases to react at room temperature for 30-60 minutes;
d. Removing impurities by filtering and centrifugation and collecting supernatant;
e. Diluting the supernatant by 50% with 95% ethanol and precipitating it at −20 to −80° C. for 12 hours;
f. Centrifuging the precipitated supernatant to collect DNA pellets;
g. Washing the DNA pellets and further collecting DNA pellets by centrifugation;
h. Drying the DNA pellets at 40 to 65° C. for 6 to 12 hours;
i. Dissolving the DNA pellets in water;
j. Treating DNA pellet solution with ultrasonic radiation (28 kHz) for 90-120 minutes to separate low molecular weight DNA (PDRN) of 100-200 bp;
k. Certifying purity (A260/A280=1.8-2.0);
l. Storing PDRN after passing it through a 0.45 m to 0.22 m filter stage.

8. The composition of polydeoxyribonucleotide (PDRN) mixture of claim 1, wherein the mixture is produced in a series of steps comprising:
a. Freezing the washed plant material in liquid nitrogen to prevent enzymatic degradation
b. Grinding the frozen plant material into a fine powder;
c. Contacting the fine powder in step (b) with a DNA extraction buffer to extract nucleic acids;
d. Mixing the buffer with proteinase K to digest proteins;
e. Precipitating protein to separate nucleic acids using cold ethanol or isopropanol;
f. Removing impurities by washing with ethanol and dissolving in nuclease-free water or a buffer;
g. Mechanically shearing by sonification or mechanical stress or using DNase I or other nucleases to reduce DNA to a smaller size;
h. Purifying the mixture by ultrafiltration or column-based separation.
